(19) United States
(12) Patent Application Publication
(10) Pub. No.: US 2008/0039867 A1
(43) Pub. Date: Feb. 14, 2008

(54) Actuator Platform For Guiding End Effectors In Minimally Invasive Interventions

(75) Inventors: Hubertus Feussner, München (DE); Eduard Sammereier, Haarbach/Rainding (DE); Martin Sellen, Ortenburg (DE); Jürgen Michael Knapp, Lappersdorf (DE); Robert Geiger, Metten (DE); Ludwig Kirschenhofer, Regensburg (DE)

Correspondence Address: HOFFMAN WASSON & GITLER, P.C;CRYSTAL CENTER 2, SUITE 522, 2461 SOUTH CLARK STREET, ARLINGTON, VA, 22202-3843, US

(73) Assignee: MICRO-EPSILON MESSTECHNIK GMBH & CO. KG, Ortenburg (DE)

(21) Appl. No.: 10/579,068

(22) PCT Filed: Nov. 5, 2004
(86) PCT No.: PCT/DE04/02442

§ 371 Date: Feb. 21, 2007

(30) Foreign Application Priority Data
Nov. 12, 2003 (DE) 103 53 110.6

(51) Int. Cl. A61B 19/00

(52) U.S. Cl. 606/130

(57) ABSTRACT

The invention relates to a universal actuator platform for guiding end effectors, for example, cameras, surgical, or medical tools, or instruments etc. in minimally invasive interventions, in which each end effector is introduced into a body cavity at an entrance point with at least one interface, for connecting at least one kinematic device, with an end effector, to at least one drive mechanism for the kinematic device and to a drive controller.

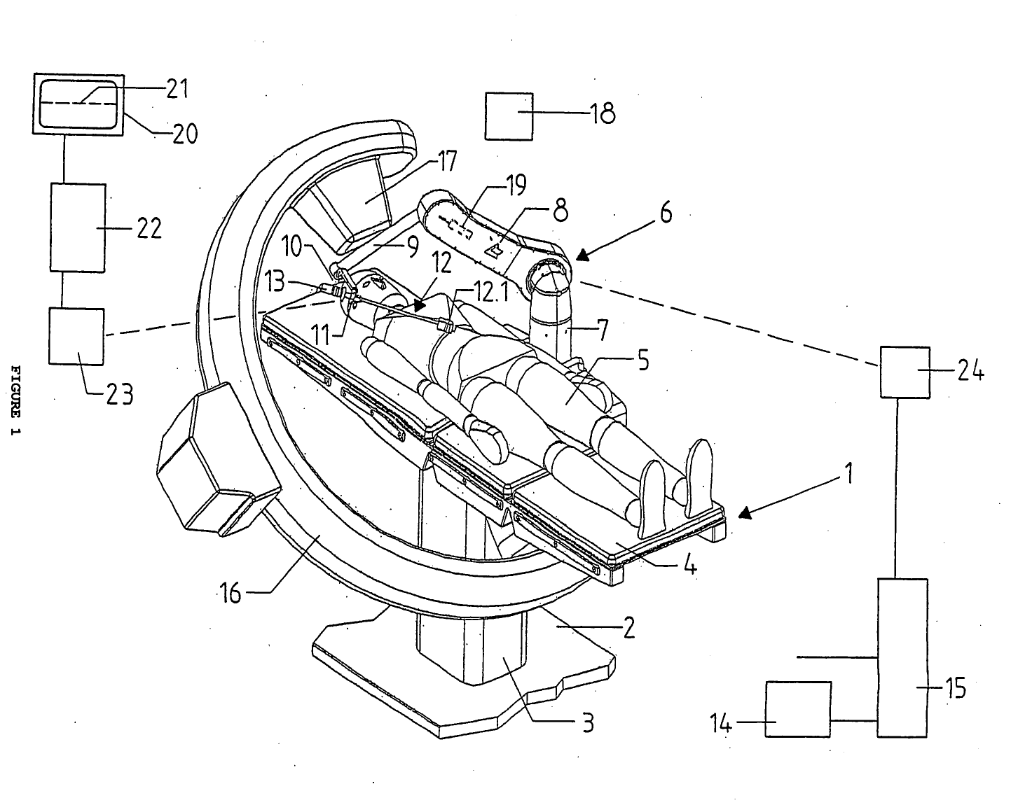

The invention relates to a robot or to a universal actor platform for guiding end effectors, for example, cameras, instruments, etc., which are inserted into the body space of a human or animal body at an entry or surgical opening for minimal invasive interventions.

Surgical assist robots or actor platforms for use in medical interventions or operations are known in the art, e.g. for guiding ancillary instruments such as cameras, etc.

Also known are minimal invasive interventions in which an instrument, for example a surgical instrument or an optical or imaging instrument, for example an endoscope, is inserted into the interior of a patient's body through a small surgical opening.

The object of the invention is to present a universal robot system or a universal actor platform for guiding end effectors during minimal invasive interventions, which (actor platform) completely fulfills the diverse and at times also contradictory requirements of daily clinical use, and which features a small and compact design with low weight and high stability, which enables without hindrance the use of a wide range of imaging systems for examinations and/or monitoring inside the human or animal body and which ensures, by means of actuators and controllers, a precise movement of the end effectors also in the event of external interfering factors.

This object is achieved with an actor platform according to claim 1.

“Imaging or image-producing media” according to the invention are, for example, X-rays, magnetic fields or electromagnetic waves from processes or systems based on these media and used in the medical field, for example X-ray machines, computer tomography systems or devices based on nuclear spin or magnetic resonance imaging, electromagnetic position indicating devices or systems, etc.

A “neutral material” according to the invention is a material, which is neutral or approximately neutral for these imaging or image-producing media, i.e. is permeable in particular to the respective medium and exhibits no or at least no significant reaction with the imaging or image-producing medium. Neutral materials in this sense are, for example, materials that are neither ferro-magnetic nor diamagnetic and are also not electrically conductive or exhibit only minimal electrical conductivity and preferably also only minimal dielectric losses.

Suitable neutral materials are, for example, plastics or also inorganic materials, such as ceramics, and possibly also soft metal alloys, such as aluminum alloys.

If the surgical assist system according to the invention is used on an operating table, then preferably at least such elements of the system located above the operating table level, including actuators, joints, etc. are made of one or more neutral materials.

The invention is described in more detail below based on an exemplary embodiment with reference to the drawing, which shows in a simplified representation an operating table with a C-arm with an imaging device and with an actor platform for guiding end effectors during a minimal invasive intervention.

The operation table generally designated 1 in the drawing consists in the known manner of a base element 2, a lifting column 3 and the actual table element 4, which forms the table surface for the patient 5 during an operation or a minimal invasive intervention.

In the depicted embodiment, a kinematic unit 6 of a universal actor platform (robot system) is connected with the operating table. In the depicted embodiment, the kinematic unit consists of a support column 7 and of a plurality of arms 8, 9 and 10, the arm 10 forming an interface or holder 11 on its free end on which an end effector 12, which in the depicted embodiment is an endoscope with a camera 13, is held so that it can be moved on several axes by means of a motor. The kinematic unit 6 forms a plurality of movement axes, on which or in which the holder 1 can be pivoted or moved.

For the operation (minimal invasive intervention), the end effector 12 is inserted with its head or with its instrument and end effector tip 12.1 (for example lens of the endoscope) through a surgical opening into the surgery area in the body of the patient 5 and can be controllably moved with corresponding actuators of the actor platform or kinematic unit 6 by the surgeon, namely by means of any type of input unit 14 of an actuator controller or electronic control unit 15. The electronic control unit 15, which is part of the actor platform, is used to control the kinematic unit so that the instrument or end effector tip 12.1 is moved in the desired manner in the body space of the patient 5, without the position of the end effector area at the surgical opening as an invariable point being changed or significantly changed during this movement.

On the operating table 1 there is furthermore a so-called C-arm 16, on which an imaging device 17, for example the radiation source of an X-ray imaging device is provided, for the purpose of an imaging examination and/or monitoring of the surgical area in the patient.

In addition to X-ray imaging devices or methods, other imaging processes or systems can also be used, such as nuclear spin or magnetic resonance imaging. Furthermore, it is possible to provide an electromagnetic position indicating device 18 for the exact determination and/or validation of the position of the instrument or end effector tip 12.1.

In order to enable an unhindered imaging examination for all standardly used processes and systems, all functional elements of the kinematic unit 6 at least above the level of the table element 4, in particular all arms 8-10, joints and actuator elements are made of a material that is neutral for the imaging processes or systems and their media, i.e. in particular of a material that is not electrically conductive and is not ferro-magnetic or dia-magnetic.

Suitable materials are for example insulating and simultaneously magnetically neutral materials, such as plastic with sufficient stability, e.g. PA (polyamide), POM or PE (Polyethylene). Aluminum alloys are also suitable to a limited degree.

Unsuitable materials for the functional elements of the kinematic unit 6 at least above the level of the table element 4 are all magnetic and/or metal materials, such as steels, in particular stainless steel, and materials with a high density.

Suitable actuators for the invention are for example actuating cylinders, e.g. hydraulic actuating cylinders, as indicated by 19 in the drawing. These actuators are then likewise made of a material that is neutral for the imaging medium.

The instrument or end effector tip 12.1 is designed for example so that it is detected by the imaging system, in order that the position of this tip is also indicated by the imaging system.

Additional, special characteristics of the universal actor platform 6 and its components are described in more detail below.

### Kinematic Unit 6

The kinematic unit is designed so that it enables a movement of the end effector 12 or of the head or tip 12.1 by 360° at the entry point of the end effector 12 formed by the respective surgical opening in the interior of the body of the patient 5, so that, as already described, the position of the area where the end effector 12 is inserted into the body at the entry point or surgical opening does not change or does not change significantly. Furthermore, the kinematic unit 6 is designed so that it enables an inclination of the end effector 12 by at least 75° from perpendicular in relation to the entry surface on which the surgical opening or entry opening is provided.

In addition to being manufactured from the neutral materials, the kinematic unit 6 must also have a small and compact design so that it does not reduce the space around the operating table 1 or the table element 4, i.e. the space required for the kinematic unit 6 corresponds even during extreme movements essentially to the space required for a human surgeon, but is preferably smaller than the space required by a human surgeon.

Furthermore, the kinematic unit must have a low weight, i.e. a total weight of less than 15 kg, so that the kinematic unit can easily and conveniently be fastened to the table element 4 or on mounting rails located there, possibly also using quick-release fasteners or quick-action clamps so that it can also be removed from the operating table.

A further essential characteristic of the kinematic unit 6 consists in the fact that it can be quickly released in an emergency, so that the respective end effector 12 can be removed manually from the body of the patient 5. The unit is released for example on at least one joint between two adjacent arms of the kinematic unit 6.

In order to effectively clean and sterilize the kinematic unit 6, it has a suitably closed design, which can be achieved for example by forming the outer surface of the kinematic unit at least in the proximity of the joints from a flexible hose.

The actuators for the kinematic unit 6 are preferably fluidic, e.g. hydraulic actuators, combining a compact design with high forces and moments, assuring in particular that the kinematic unit 6 is highly stable. In addition, the fluid actuators also enable slow and precise movements.

In order to use the kinematic unit 6 with systems based on magnetic resonance and/or X-rays and/or electromagnetic fields, the actuators present in the kinematic unit are preferably secondary or slave actuators, which are located outside the sphere of influence of the magnetic resonance, X-rays or electromagnetic fields. The primary actuators are for example pumps or master cylinders. The secondary actuators are for example cylinders.

### Actuator Controller or Electronic Control Unit 15

A further component of the universal actor platform is the electronic control unit 15, which enables very simple control of the kinematic unit 6 via the input unit 14 (for target values) in small increments. The input unit 14 can have any design whatsoever and enables for example the manual control or manual input of control commands. It is fundamentally possible to provide this input unit 14 on medical instruments used by the surgeon in addition to the end effector 12 attached to the kinematic unit 6. Other control or input units are also possible, for example an automatic control of imaging elements or systems and/or of sensor elements (e.g. on the head 12.1 of the end effector 12). Voice control is also possible.

The values entered via the input unit 14 are then compared as set values with actual values provided by sensors, defining the current status or the current position of the kinematic unit 6, thus enabling very exact positioning and movement of the end effector 12 through a closed control loop.

The electronic control unit 15 preferably also features further characteristics. For example, the electronic control unit, possibly in combination with external sensors, achieves an interference compensation in the control of the kinematic unit 6, in particular a compensation of external mechanical impacts or vibrations on the operating table 1 and a compensation of electric and electromagnetic interference and/or influences of temperature.

Furthermore, the kinematic unit 6 is controlled by the electronic control unit 15 so that a plausibility check precedes the introduction of a movement or change of position, e.g. by comparing the current position of the end effector 12 or of the head 12.1 with the respective input, wherein obstacles are also automatically detected and bypassed for example by moving the end effector 12. Furthermore, the sphere of movement of the end effector 12 or of the head 12.1 is limited.

In a preferred embodiment of the invention the electronic control unit is designed so that tracking optimization of the kinematic unit 6 takes place when it is not moving. In this process, the state of the individual movement axes of the kinematic unit 6 is optimized without moving the end effector 12, i.e. maintaining the current position of said end effector, so that from the state then achieved, each movement axis can execute the assigned movements for a corresponding command without limitations, i.e. so that no movement axis is located in an end position.

Furthermore, the electronic control unit is preferably designed so that the movement of the end effector 12 effected by input takes place on an optimum, short path of motion and/or within a small sphere of movement of the kinematic unit, thus likewise minimizing the space required for the kinematic unit 6.

The electronic control unit 15 can be connected with additional external devices by means of adaptable interfaces, e.g. for voice control, etc.

In the depicted embodiment, the instrument 12 with the camera 13 can be turned freely or essentially freely on the instrument's longitudinal axis at the free end of the arm 10 or of the instrument holder 11, so that the instrument 12, when positioned at an angle for example, rolls off with its circumference on the edge of the body opening, thus turning on its axis, when pivoting on the axis of the invariable point or of the body opening on which the instrument 12 is inserted into the body of the patient 5. This would mean that the camera image, or its image horizon indicated in the drawing by 21, recorded by the camera 13 and displayed on a monitor 20 would also turn, thus making it very difficult for the surgeon to visually evaluate the camera image.

To prevent this difficulty, the image provided by the camera 13 is processed in an image processor 22 so that even if the instrument 12 is pivoted on the axis of the body opening and the instrument 12 then turns on its instrument axis, at least the alignment of the image or of the image horizon 21 remains unchanged or essentially unchanged. For this purpose, the image processor 22, which of course can also be a component of the electronic control unit 15 or of a corresponding computer or of a software, is connected with a sensor 23, which is provided on the arm 10 or on the instrument holder 11 and provides a sensor signal, which corresponds to the turning position of the instrument 12 or of the camera 13 on the instrument axis relative to the arm 10. This sensor signal is then used to process or turn the image provided by the camera 13 so that at least the image horizon 21 retains its orientation.

Furthermore, it is possible to perform this correction by means of an intelligent image processor, so that the position of the image provided by the camera 13 is corrected in the image processor 22, for example based on clearly defined image elements and/or based on additional image elements produced in the camera, which exhibit a fixed position in the image plane of the camera 13.

Since the kinematic unit 6 features sensors, with which the respective position of the kinematic unit 6 and its movement in or on the kinematic axes is recorded and from which the position and orientation of the instrument 12 and of the camera 13 can be calculated, e.g. by the control unit 15, a further possibility exists for maintaining at least the orientation of the image horizon 21 by correcting the image provided by the camera 13 based on the signals provided by said sensors. The sensors, depicted schematically in the drawing, are designated 24.

If the actuating cylinders 19 are, as mentioned above, slave cylinders of a slave-master system in which each actuating cylinder 19 is actuated by one control cylinder via a fluid connection (hydraulic connection), then the sensors 24 and the control cylinders are located outside of the sphere of influence of the magnetic resonance, X-rays and/or electric fields.

It was assumed above that the image or image signal provided by the camera 13 is corrected in order to maintain the orientation of the image horizon 21. It is also possible to maintain the orientation of the image horizon 21 by having an actuator turn the camera 13 for example together with the instrument 12 on the axis of said instrument, preferably automatically, e.g. using the signals provided by the sensors 24 of the kinematic unit.

The invention was described above based on one exemplary embodiment. It goes without saying that numerous modifications and variations are possible without abandoning the underlying inventive idea upon which the invention is based.

### REFERENCE LIST

1 operating table
2 base element
3 lifting column
4 table element
5 patient
6 kinematic unit
7 support column
8-10 arm
11 instrument holder
12 instrument
12.1 lens
13 camera
14 input unit
15 electronic control unit
16 C-arm
17 element or radiation source for imaging device
18 electromagnetic position indication device, actuator
19 actuating cylinder
20 monitor
21 image horizon
22 image processor
23, 24 sensor

What is claimed is:

1. A universal actor platform for guiding of end effectors, cameras, surgical or medical instruments, during minimal invasive interventions, in which the respective end effector is inserted into an interior body space at an entry point, with at least one interface for connecting a kinematic unit comprising at least one end effector, with at least one actuator for the kinematic unit and with one actuator controller wherein the actor platform comprises in combination:
the kinematic unit enables movement of the at least one end effector by 360° at the entry point and an inclination of at least 75° from perpendicular in relation to the plane of the entry point;
the kinematic unit comprises at least in a partial area of a material that is system-neutral for the medium of an imaging and/or position and/or orientation indicating device;
the kinematic unit further comprises means for fastening to an operating table or mounting elements, or rails, located there;
the kinematic unit further comprises a closed design, enabling sterilization;
the kinematic unit can be manually released or removed;
the actuator controller comprises an input unit for target values;
the actuator controller is designed for compensation of interference factors, in mechanical, electrical or temperature-related interference factors.

2. The universal actor platform according to claim 1, wherein the kinematic unit can be manually released or removed on at least one joint.

3. The universal actor platform according to claim 1, wherein the kinematic unit has a small design with low space requirements and/or has a weight of less than 15 kg.

4. The universal actor platform according to claim 1, wherein the kinematic unit can be fastened laterally on one side, on one longitudinal side of the operating table or on mounting elements, rails, located there.

5. The universal actor platform according to claim 1, wherein the actuator controller, in order to achieve high accuracy and reliability, is part of a closed control loop, in which the at least one actuator is controlled by comparing the actual value provided by at least one sensor and representing the actual state of the kinematic unit with a set value.

6. The universal actor platform according to claim 1, wherein the actuator controller is designed so that the movement of the at least one end effector is controlled by the actuator controller taking into account a plausibility check and/or within a limited sphere of movement.

7. The universal actor platform according to claim 1, wherein the actuator controller is designed so that the movement of the at least one end effector is controlled by the actuator controller on an optimum path of motion, on very short paths and/or within a very small sphere of motion.

8. The universal actor platform according to claim 1, wherein the kinematic unit and its elements are made of the neutral material at least in one element above the level of the table surface for the patient.

9. The universal actor platform according to claim 1, wherein the kinematic unit can be fastened to the operating table or the table element by means of at least one quick-release connector.

10. The universal actor platform according to claim 1, wherein the at least one actuator of the kinematic unit is a fluid actuator, an actuator with at least one master element and one slave element.

11. The universal actor platform according to claim 1, whereby a simple input of set values and/or control is in small increments.

12. The universal actor platform according to claim 1, further comprising means for the manual input of control commands, through manually actuated input means, to a medical instrument.

13. The universal actor platform according to claim 1, further comprising means for the automatic input or control of the kinematic unit via imaging elements and/or sensor elements.

14. The universal actor platform according to claim 1, further comprising means for the voice control of the actuator controller and kinematic unit.

15. The universal actor platform according to claim 1, wherein the actuator controller is designed for tracking optimization of the kinematic unit when the end effector is not in motion, and that the kinematic unit can be controlled for this tracking optimization by the actuating controller so that when the end effector is not in motion the individual movement axes of the kinematic unit are set so that after making the setting, each movement axis is in a state from which the largest possible movement stroke of the movement allocated to the respective axis is possible.

16. The universal actor platform according to claim 1, wherein when the end effector is a camera or part of a camera, means are provided to keep the position and/or orientation of a camera image reproduced on a monitor constant or approximately constant.
